(19) United States
(12) Patent Application Publication
(10) Pub. No.: US 2003/0229278 A1
(43) Pub. Date: Dec. 11, 2003

(54) Method and system for knowledge extraction from image data

(76) Inventor: Usha Sinha, Los Angeles, CA (US)

Correspondence Address: Frank J. Bozzo, Esq., DORSEY & WHITNEY LLP, Suite 3400, 1420 Fifth Avenue, Seattle, WA, 98101, US

(21) Appl. No.: 10/165,674

(22) Filed: Jun. 6, 2002

(51) Int. Cl. A61B 5/05; G06F 17/00

(52) U.S. Cl. 600/407; 128/920; 382/128; 600/410; 600/425

(57) ABSTRACT

A method and system are described that identify anatomical abnormalities in internal images of a subject under study. The method and system use principal component analysis of a subject's image as compared to a training set of images. The training set of images incorporates both normal and abnormal cases. Specifically the principal component analysis identifies key image slices to pinpoint image slices whose vectorized and transformed representations quantitatively diverge from training set images identified as normal and/or resemble training set images identified as abnormal. The method and system automatically classifies images as normal or abnormal based upon the content of the images, and/or automatically provides comparable reference images for aiding physicians in reaching a diagnosis.

### TECHNICAL FIELD

The present invention is directed to the analysis of image data generated through imaging technologies such as magnetic resonance imaging and computed tomography scanning. More particularly, the present invention is related to a method and system for automating the identification of normal and abnormal images representing target patients with reference images showing previously diagnosed lesions and disease states.

### BACKGROUND OF THE INVENTION

Medical imaging techniques, such as magnetic resonance imaging (“MRI”) and computed tomography scanning (“CT scanning”), have become predominant diagnostic tools. In fact, these techniques have become so prevalent that their popular abbreviations, “CT scan” and “MRI,” respectively, have literally become household words. Effective diagnosis of a multitude of medical conditions, ranging from basic sports injuries to the most costly and pressing health care issues of today, including cancer, stroke, and heart disease, would be far more difficult, if not virtually impossible, without these imaging technologies.

These technologies allow medical professionals and researchers to literally see what is happening inside of a patient in great detail without resorting to invasive surgery. Magnetic resonance imaging, for example, generates a series of two-dimensional view slices of a patient in any of sagittal, coronal, or axial cross-sectional views. A series of these views represent in three dimensions a patient's complete internal anatomy and physiology. By studying a patient's images and comparing them with known references that exemplify images of abnormal conditions (e.g., presence of a brain tumor), physicians and other health care professionals can be assisted by the computer to make more accurate diagnosis and better assess the response of a disease to a therapy by comparing to previously treated patients with known disease and outcome.

For example, FIGS. 1A, 1B, and 1C show three simplified axial imaging slices of a human brain derived from an imaging study. Imaging slice 100 of FIG. 1A depicts a normal brain 110, free of abnormal lesions. Imaging slice 120 of FIG. 1B, by contrast, depicts a brain 130 which afflicted by a relatively large lesion 140. Image slice 150 of FIG. 1C depicts a brain 160 afflicted by a very small lesion 170. When patients with any of these lesions is operated on or the lesion is biopsied and the content is examined by microscope, then a definitive diagnosis can be assigned to a lesion (e.g., diagnosis of a specific type of brain tumor). Physicians gain experience with time and can gradually become experts and experience based on their own encounters of patients with various medical conditions. However, no physician can have sufficient experience with all medical conditions.

One problem with the ever-expanding use of medical images is the effective use and management of the overwhelming volume of data generated by these technologies. As with other computer graphics applications, medical imaging generates huge quantities of data. A typical imaging study can range, for example, anywhere from 13 megabytes to 130 megabytes in size. Moreover, with improvements in imaging resolution, these quantities are expected to increase. Merely storing these great quantities of data may not be a tremendous concern because of the increasing density and price performance of data storage devices.

A separate, greater concern is the amount of time required to effectively analyze these enormous bodies of data. FIG. 2 depicts just a subset of the axial image slices taken from the brain of one single patient. FIG. 2 depicts just sixteen different image slices from an imaging study of a human brain; a typical brain imaging study can comprise sixty or more different image slices. FIG. 2, in which the image slices might be millimeters apart, portray subtly different views of the brain, and careful review of all the many axial image slices would be very time-consuming. Nonetheless, for the reasons discussed, such careful review of every image slice is very important.

In today's information processing environment, it is a simple matter for a computer to analyze basic medical information that can be reduced to scalar quantities such as temperatures, heart rates, and blood chemistry information. Once a patient's vital statistics are entered in a computer system, the system can compare those statistics to what would be expected and automatically identify patients with fever or whose heart rate and blood pressures indicate hypertension and/or other cardiovascular disorders. However, it is an entirely different problem for a computer to analyze the dozens of images that might be generated in a single medical imaging session when one considers that any single graphical image may comprise as large a body of digital data as the textual and numerical medical history of many patients. Numerically analyzing even a few medical images would exceed the capacity of many computing systems using known methodologies. What is needed as a more efficient way to represent of data reflected by these medical images to provide for their affective computer analysis.

Some computer systems have been used in this context in order to assist physicians in identifying abnormal imaging slices. However, each of these systems have significant limitations. Some of these systems require a physician such as a radiologist to manually select images from a specific region of interest. To make an educated comparison with images acquired from his patient, the physician might have to manually and carefully study all the images, for every single image slice, to even be able to identify possible abnormalities and, thus, the region of interest. Accordingly, such a system saves the professional little or none of his or her valuable time, and does little or nothing to support his or her initial study of a patient's medical condition.

Present, so-called automated systems in use also require a great deal of human expert involvement. For example, some systems allow an expert to textually describe images of a known condition, then subsequent users can search these textual records to find images which may correspond to the patient images of interest. Obviously, such a system is limited in that it requires an expert to do a great deal of work to catalog and characterize the images, it requires the physician accessing the system take the time to frame a workable query, and if the query posed by physician accessing the system does not use the same syntax as that of the expert, the query may yield no helpful results even if appropriate entries exist in the database. What is needed as a more effective and cost effective way to value it medical images to ensure early detection of disease and prescription of proper treatment.

Benefits of an effective system for autonomously analyzing and classifying medical imaging data extend far beyond that of benefits just to the patients being diagnosed. Once meaningfully classified, the imaging data can be put to good use by many other people. Just for example, images classified as representing certain diseases or other problems could be easily retrieved and studied by physician teachers and students, medical researchers, and other professionals. It can also be used as an effective decision support system for any health care provider.

At present medical imaging data is studied manually, with radiologists and other trained medical personnel visually inspecting the image data collected to actually look for abnormalities. Physicians can then make their diagnosis based on their own past experience or by manually comparing patient images with the textbooks. What truly is needed is a way to take advantage of computer technology to screen and prescreen imaging data for diagnostic purposes as well as to assist radiologists and other medical professionals in studying, in researching, and diagnosing injuries and illnesses. Saving the time of medical professionals, providing those professionals with better training, and enhancing the possibility of early diagnosis of disease are just a few of the benefits of such a system that can improve countless lives and reducing presently skyrocketing health care costs. It is to these ends that the present invention is directed.

### SUMMARY OF THE INVENTION

The present invention comprises a method and system to identify anatomical abnormalities in internal images of a subject under study, which comprise query images. The present invention uses principal component analysis of query images to compare them to a basis set of training images. The training images incorporate both normal and abnormal cases having previously confirmed diagnoses. Specifically the principal component analysis identifies key query images to pinpoint image slices whose vectorized and transformed representations quantitatively diverge from training images identified as normal and/or resemble training images identified as abnormal. The method and system classifies query images or selectively identifies comparable training images based solely upon the content of the query images as compared to the training images, and does not rely on supplemental information entered by individuals reviewing the image data.

The method and system disclosed in the disclosed embodiments involves four general processes. The first process is the collection of the training images. The second process is the calculation of an eigenspace defined by the training images. The third process is the standardization of image aspects, including orientation, contrast, and other factors, to facilitate comparison of new images to the database of the training images. The fourth process is dependent upon whether the present invention is used for image summarization or classification or used for decision support. For image summarization, the fourth process is the classification of query images as either normal or abnormal based on automated comparison with the basis set comprised of the training images. For decision support, the fourth process is the identification of the closest matching image from the training images for a physician to use compare with the query image or images, assisting the physician in diagnosing the patient.

### BRIEF DESCRIPTION OF THE DRAWINGS

FIG. 1A is an axial view of a human brain that exhibits no abnormalities.

FIG. 1B is an axial view of a human brain that is afflicted with a large abnormality.

FIG. 1C is an axial view of a human brain that is afflicted with a small abnormality.

FIG. 2 is a minor subset of a series of axial views of a brain taken at different points along an axis collinear with the patient's spine.

FIG. 3 is a flowchart of the overall process employed in creating a database of training set images and using that database to analyze images derived from a patient's imaging study in a preferred embodiment of the present invention.

FIG. 4A is an axial image of a human brain presented with low image intensity and a histogram representing the intensity level.

FIG. 4B is an axial image of a human brain presented with higher image intensity and a histogram reflecting the intensity level

FIG. 5 is a subset of a series of axial views of a brain with corresponding eigenimages generated by and used in an embodiment of the present invention.

FIG. 6 is a flowchart of the process employed in classifying images derived from a patient's imaging study.

FIG. 7 is a block diagram of an embodiment of a system of the present invention.

FIG. 8 is a block diagram of an alternative embodiment of a system of the present invention.

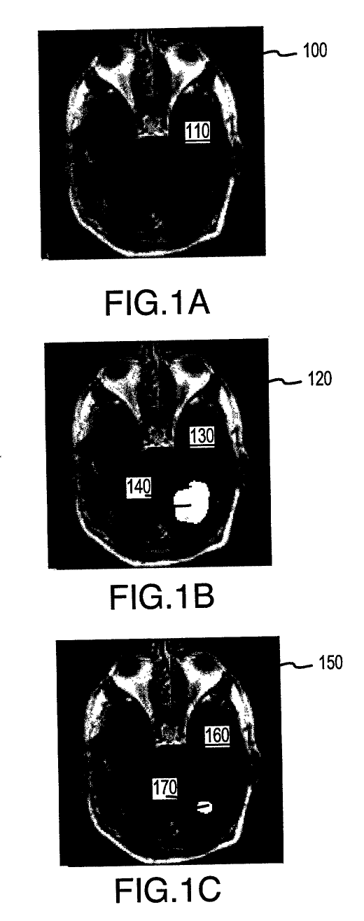

### DETAILED DESCRIPTION OF THE INVENTION

It will be appreciated that embodiments of the method and system of the present invention can be used for any region of a subject's anatomy, such as the pelvis, extremities, or any other region that is rigid. Further discussion of the embodiments of the present invention, for illustration, will use the example of internal imaging of a human brain. Moreover, the subjects could be human, animal, or another entity from which professionals in that field could benefit from automatic classification of internal imaging studies to identify normal and/or abnormal images. Embodiments of the present invention can be used with images acquired with magnetic resonance imaging, computed tomography scanning, or other techniques.

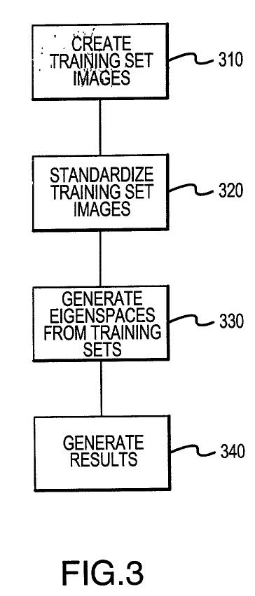

Generally speaking, embodiments of the present invention use a database of mathematical representations of training images to evaluate and classify a query image of a patient as normal or abnormal. More specifically, as shown in FIG. 3, one embodiment of the present invention for image summarization uses four processes: creation of a basis set of training images 310; standardization of the training images to facilitate comparison with new images 320; calculation of eigenspaces representing the training images 330; and generating a result 340. At 340 one embodiment of the present invention can be used to automatically classify query images as either normal or abnormal based on their quantitative vectorized comparison to the eigenspaces derived from the basis set of training images. Alternatively at 340 an embodiment of the present invention can be used to automatically identify training images which are most comparable to the query image, providing decision support to a medical professional performing a manual evaluation of the query image.

The first step is creating the training images 310 is actually collecting or compiling the basis set of training images. Training images are archival images collected from other patients which eventually will be compared with query images, which are the presently acquired images of a target patient. Eventually, in the classification process 340 (FIG. 3), representations of the query images will be compared with representations of the training images to evaluate whether the query images exhibit any abnormalities.

The training set images are selected and classified manually by persons with expertise in reviewing patient images and diagnosing abnormalities. Images ideally should be selected so as to represent a wide cross-section of different types of both normal and abnormal images. Both normal and abnormal images are used in a preferred embodiment to improve the identification accuracy; images of different patients, whether normal or abnormal, will appear identical, and the process of identifying abnormal images involves a relative comparison from among the training images.

Training images should be assembled for every location in the chosen region of the anatomy with which the system will be used to classify images. For example, the region might be the brain and the location comprises images within five millimeters of the third ventricle. A preferred embodiment would include a minimum of fifteen sets of training images for each anatomical location with, for example, coverage of an area of ten millimeters by ten millimeters in size to cover the span of the human brain. A target of one hundred images per set of training images is desirable, with forty percent comprising images which persons with expertise have identified as abnormal, and sixty percent comprising images which persons with expertise have identified as normal.

The preferred embodiment employs human expert involvement in collecting and classifying the training images. However, unlike other pre-existing techniques in which human expert involvement is required in additional steps, human involvement is not further required in embodiments of the present invention.

After the training images for desired locations for image analysis have been compiled at 310, the second step is to standardize the collected images at 320. To perform a meaningful comparison of new images against the collection of training images, the images must be standardized to eliminate nonsubstantive variations between images stemming from differing levels of illumination, orientation, image intensity, size and similar factors resulting from the circumstances under which the images were captured and recorded. Uniformity in image acquisition eliminates the need for this step if sufficient uniformly acquired images are available to create sufficient training image sets. However, being able to only use uniformly acquired images would greatly limit the supply of possible images from which training image sets can be drawn. Thus, it is desirable to be able to standardize images acquired under nonuniform conditions.

Differences in image intensity pose a particular concern. Standardization of image intensity requires preprocessing of image data. In a preferred embodiment, necessary preprocessing would be completely automated. Further, in a preferred embodiment, the preprocessing would be as computationally non-intensive as possible to reduce the computing resources and/or computing time needed to process images. Alternative embodiments could use standardization of contrast and/or contrast insensitive measuring to standardize image intensity. One such method and system for standardizing such images is described in concurrently filed U.S. patent application Ser. No. ______ by Sinha entitled “METHOD AND SYSTEM FOR PREPARATION OF CUSTOMIZED IMAGING ATLAS AND REGISTRATION WITH PATIENT IMAGES.”

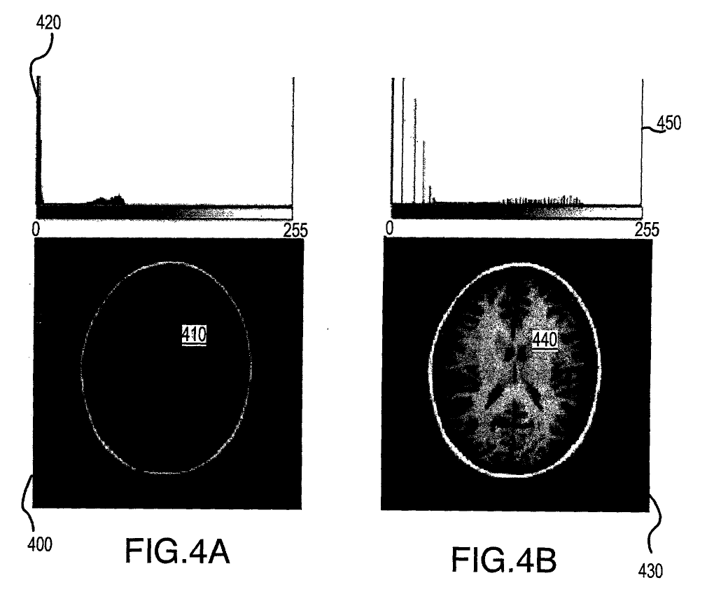

In one embodiment, standardization of contrast can be performed by creating a histogram of an image and equalizing the pixel intensity of the histogram. Histogram equalization is a mathematical process that increases the contrast in the image by spreading the pixel distribution equally among all the available intensities. This results in a flatter histogram for each image. FIG. 4A shows a sample image slice 400 of a brain 410 and an associated histogram 420 representing the intensity of the image slice 400. The horizontal axis of the histogram 420 reflects pixel intensity level, and the vertical axis reflects a number of pixels. Accordingly, the histogram reflects the number of pixels represented at each pixel density level. FIG. 4B shows an adjusted image slice 430 of the brain 440 and a histogram 450 representing the intensity of the image slice 430 after equalization. Each image was scaled to range between 0 and 255, so as to have a common dynamic range for the images from different subjects.

As shown in the histogram 420 of the original image slice 400 shown in FIG. 4A, most pixels are clustered around the lower grayscale intensities with loss of image detail. Histogram equalization was performed to obtain similar contrast enhancement in the image sets. Beginning with the pixel density represented on the original histogram 420 derived from the image slice 400, pixel intensity is redistributed to generate a flatter histogram 440, with a more even distribution of pixels at each density. The new image slice 430 is regenerated in accordance with this flatter histogram 440. As a result, the histogram-equalized image slice 430 shown in FIG. 4B clearly shows greater image detail, especially in the dark portions of the image slice 430. The histogram 450 for the adjusted image slice 430—which, again, was not derived from the image slice 430 but was used to adjust the image slice 400 to make image slice 430—clearly shows the spread of the pixel intensity values as a result of the histogram equalization.

It should be noted that pixel intensity or contrast redistribution is not the only means of rendering the image slices to allow for comparative study of reference and target images. Alternatively, contrast insensitive measurements could be employed in an eigenimage matching algorithm, which will be described below.

Image intensity ratios are less sensitive to scaling differences between training and query image sets. In order to reduce the bias introduced by noise pixels, the logarithm of the ratios were taken with the covariance matrix O now given by the inner product of image vectors

$$log ( \frac{{{x}^{->}}_{i}}{m} ) and log ( \frac{{{x}^{->}}_{j}}{m} ) .$$

To eliminate the salt-pepper appearance of the background, all pixels that were below a threshold value in the average image were set to zero in the log-ratio image.

Not only must the images be standardized for image intensity, but they all must be standardized in spatial orientation and image scale. In a preferred embodiment, standardization of these parameters can be accomplished using automated three-dimensional registration of the training set images and query images. For example, the Automated Image Registration (AIR) program, version 3.0, of Woods et. al. can be used to bring all the image volumes into a common frame of alignment. The algorithm used by the AIR program requires minimal user intervention, and is based on matching of voxel intensities and has been tested for accuracy using both inter- and intra-subject registration. The registration program generates a matrix containing translation, rotation, and scaling parameters to register to a reference standard image volume. Reference to this matrix thereby ensures that all training set images and query images can be aligned and scaled to a common parameters.

Once the database of training images has been compiled and standardized, the third step is to perform principle component analysis to create basis image sets representative of the training images at 330. The basis image sets generated are eigenimages, which constitute a quantitative representation of the vectorized two-dimensional training images. These eigenimages can represent relatively large image representations in a much more compact form. For example, an imaging study whose data would require, for example, 13 to 40 megabytes can be represented by eigenimages consuming only 0.5 to 1 megabytes of storage.

An image can be viewed as a vector by concatenating the rows of the image one after another. If the image has square dimensions of L×L pixels, then the vector is of size L squared For example, for a typical image 256×256 pixels in size, the vector length or dimensionality is 256 squared, or 65,536. Each new image has a different vector and a collection of images will occupy a certain region in a extremely high dimensional space. In other words, these concatenated vectors are very large, and consume a great deal of data storage space. Moreover, the task of comparing images in this hundred thousand-dimension space is a formidable one.

The brain image vectors are large because they belong to a vector space that is not optimal for image description. The knowledge of brain anatomy provides us with information about underlying similarities of brain images from different subjects: an elliptical shape, essentially three tissue types: gray, white matter and cerebrospinal fluid. It is the presence of these similarities that permit the large image brain vectors to be reduced to a smaller dimensionality. Principal component analysis is used to render a representation for the image vectors to reduce the dimensionality of the image vectors, which facilitates efficient image indexing and searching.

Principal components analysis is used to transform a set of training images N, are represented as vectors of length L×L, where L is the number of pixels in the x (y) direction. The average image, m, of the N training images is given by

$$m = \frac{1}{N} {\sum }_{i = 1}^{N} {{x}^{->}}_{i} ,$$

where \vec{x}ᵢ is the L×L dimension vector corresponding to the i^{th} image in the training set. An N×N matrix called the covariance matrix O, is formed whose elements Oᵢⱼ are given by the inner product of image vectors (\vec{x}ᵢ−m) and (\vec{x}ⱼ−m). Identifying νₙ and λₙ as the eigenvectors and the eigenvalues of the covariance matrix O, respectively, there will be N−1 eigenvectors of length N. These eigenvectors determine linear combinations of the N training images to form the basis set of images, uᵢ, that best describe the variations in the training images:

$${{u}^{->}}_{i} = {\sum }_{k = 1}^{N} {v}_{ik} ( {{x}^{->}}_{k} - m ) ;$$

for i=1,2, . . . N.

The resulting eigenimages with the largest eigenvalues contain the most information in some sense and can be thought of as prototypical images. Each image in the set can then be approximated with a linear combination of these eigenimages,

$${x}_{k} \approx {Σ}_{p} {w}_{p} {u}_{p} .$$

The coefficients wₚ are projection coefficients which are calculated for each image in the set of training images. The coefficient wₚ is the feature description for the image xₖ, each of which is assigned to a different class “k.” Projection coefficients of images on the basis set of training images will be calculated for each training image. These projection coefficients specify a unique signature for an image, thus a 256×256 image vector can be uniquely specified by one hundred coefficients.

Ultimately, brain images are represented as a weighted combination of eigenimages that are derived from the training images. The eigenimages are ordered, each one accounting for a different amount of the variation among the images. These eigenimages can be thought of as a set of features that together characterize the variation among the images. The space spanned by the eigenimages is called the eigenspace. Each image location contributes more or less to each eigenimage, so that the eigenimage appears like a ghostly brain that can be termed an “eigenbrain.” Each eigenbrain deviates from uniform gray where some feature differs among the set of training images. In other words, each eigenbrain represents a map of the variations between the images.

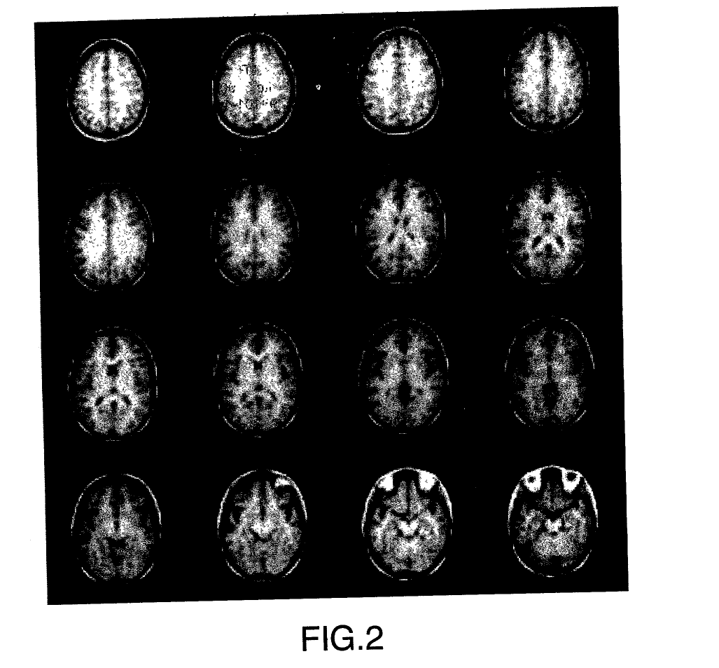

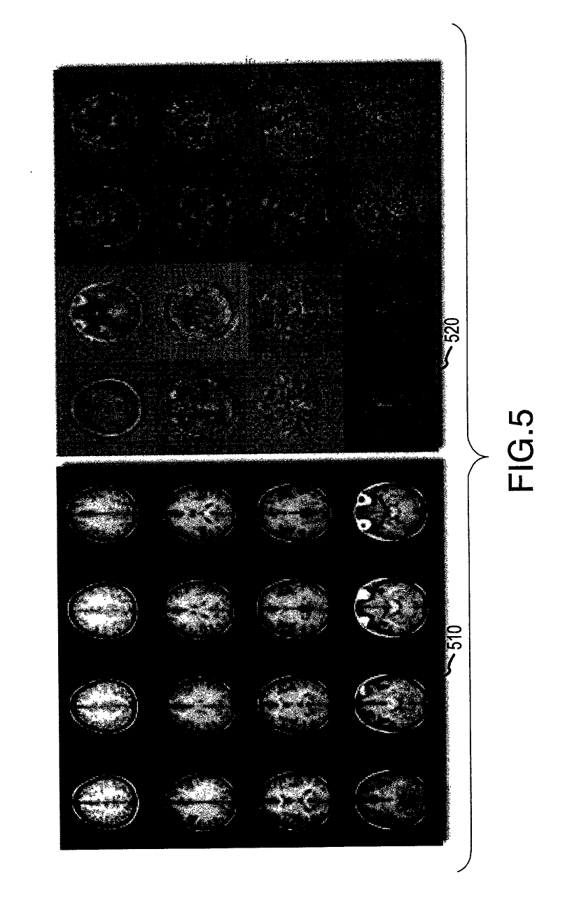

FIG. 5 shows a subset of sixteen images 510 from an axial image study of a brain. In fact, the subset of the basis set of training images 510 is the same subset shown in FIG. 2. FIG. 5 also shows a visual representation of sixteen eigenimages 520, or “eigenbrains,” derived from those sixteen slices of training set images 510 using principal component analysis. As shown in FIG. 5, the eigenimages 520 capture variations contained within the training set images 510. Moreover, it can also be seen that images in the bottom row of the training images 510 are dominated by noise and, thus, have less image content. As a result, the corresponding eigenimages have lower eigenvalues, as reflected in the relative lack of image content as shown in the visual representation of those eigenimages.

In the last step, after the training image sets have been collected, standardized, and transformed into eigenspaces, results are generated at 340 (FIG. 3). The last, result step at 340 could take the form of one of two processes in the disclosed embodiments. In an embodiment targeted for automated image study classification or summarization, the last step would result in identifying the query images as normal or abnormal. In an embodiment directed to decision support, the last step would be identifying from among the training images one or more images most closely matching the query image to allow the physician to make his or her own comparisons.

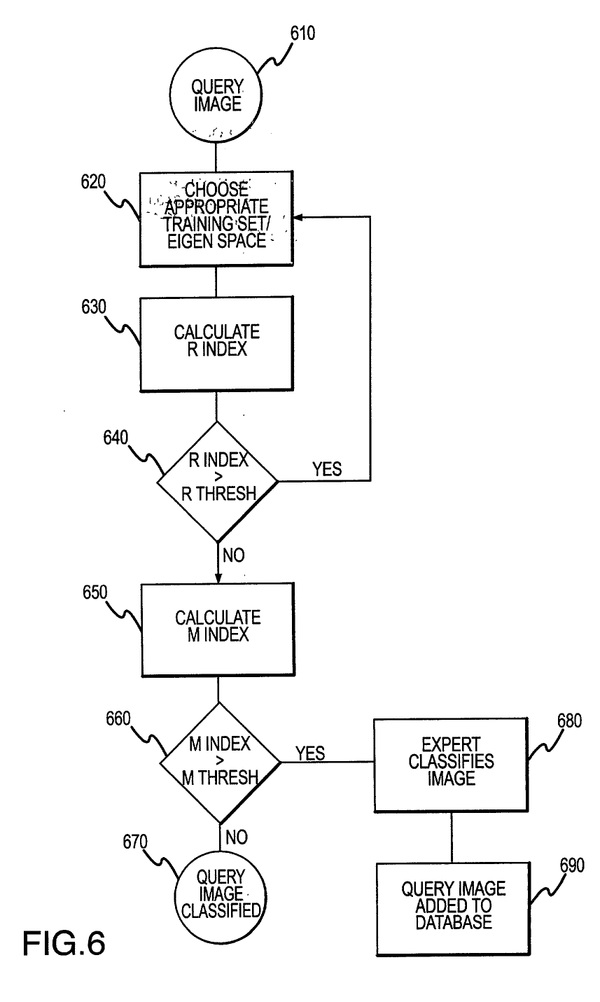

In both embodiments, query images are processed in a manner similar to that of the training images. The query image is vectorized and transformed into an eigenimage, and once the eigenspace corresponding to the suitable class of basis images has been identified, comparison of the patient images to the training images at 340 (FIG. 3) becomes a matter of mathematical computation. For automated summarization, a matching algorithm is used to determine if the query image coincides more with training images which have been classified as normal or training set images which have been classified as abnormal. Alternatively, for decision support, the matching algorithm identifies the closest image match from the training set. More precisely, of course, the eigenimage representation of the query image is compared by the matching algorithm to the eigenspace representing the training images for the location of the query image. FIG. 6 depicts the steps used in the classification process 340 (FIG. 3).

Starting with a query image or “Qimage” at 610, the first step is identifying the relevant eigenspace at 620, which can be thought of as identifying the most appropriate training set for comparison to the query image. The appropriate training set or eigenspace is that which is most nearly identical in location to that of the query image.

Choosing the appropriate eigenspace 620 is an automated process which involves determining which training set covers the region closest in location to the query image. This determination begins with the computation of the coefficient w_{q} for the query image. The coefficient w_{q} is determined by a comparison of the Euclidean distance of the coefficients w_{q} and wₚ, where p=1 through k, for k classes of the original sets of training images which were transformed into eigenspaces. If a class from a suitably close location is identified, the query image is analyzed against the eigenspace derived from that class. On the other hand, if no eigenspace representing a set of training images suitably proximate to the location of the query image exists, the query image can be used as the initial training image for a new class. It will be appreciated that embodiments of the present invention can be adapted to incorporate query images into the assembled body of training images, making the database even more comprehensive and useful over time.

The matching algorithm used to determine the proximate, appropriate training images uses two indices to evaluate whether the representation of the patient images resembles more closely the representations of training images: “Rindex” and “Mindex.” Rindex, computed at 630, is a measure of the closeness of the query image to the basis set of eigenimages of the training images, or, in other words, how well the basis image set can represent the new image. For example, the basis image set for a frontal lobe of the brain cannot serve as an adequate basis set for a query image from a different region of the brain. The quantity Rindex thus represents a quantitative measure of how well the basis image set can represent the new query image. In other words, Rindex represents a residual or the reconstruction error, and indicates whether the query image can be defined by the current eigenspace spanned by the chosen basis set. Rindex is compared to an empirically predetermined threshold value, RThresh, at 640. If Rindex is greater than Rthresh, then the query image cannot be described sufficiently well by the chosen eigenspace, and another attempt is made to identify the appropriate eigenspace.

Mathematically, if the projection coefficients of the query image are w_{q}, then the reconstructed query image, x′_{q}, is given by

$${x}_{p}^{′} \approx {Σ}_{q} {w}_{q} {u}_{q} .$$

Rindex is then defined as: RIndex²=∥x_{q}−x′_{q}∥².

If Rindex is determined at 640 to be less than Rthresh, the next step is to calculate Mindex at 650. Mindex represents the closest match between the query image and a training set image. Mindex is a measure of the closeness in the eigenspace of the query image to the closest “match image” in the basis set. Mindex is computed as the Euclidean distance between the projection coefficients of the query image and the and match image. Thus, the object of Mindex is to determine which image in the basis image that minimizes the quantity Mindex.

Mindex is compared to an empirically predetermined threshold value, MThresh, at 660. If Mindex is greater than Mthresh, then there is no image in the training set close enough to the query image to permit the query image to be classified. Both threshold values, RThresh and MThresh, can be determined empirically using a wide range of query images.

If Mindex exceeds MThresh, it implies that though the query image can be described by the eigenspace, there is no image in the training set that matches this image. At 680 An expert will then determine if the query image should be added to the training set at 690, and the expert will classify the image as normal or abnormal. It is possible, in the initial stages of implementation, that many query images may have to be included in the training set. However, as the images in the training set grow, it is anticipated that most of the variations in normal physiology as well as in pathology will be represented by the images in the training set. On the other hand, if at 660 it is determined that MIndex is less than MThresh, then the label of the closest match image, Mimage, whether that label is normal or abnormal, is assigned to the query image at 670. In other words, the output of the matching algorithm module is an image classified as normal or abnormal.

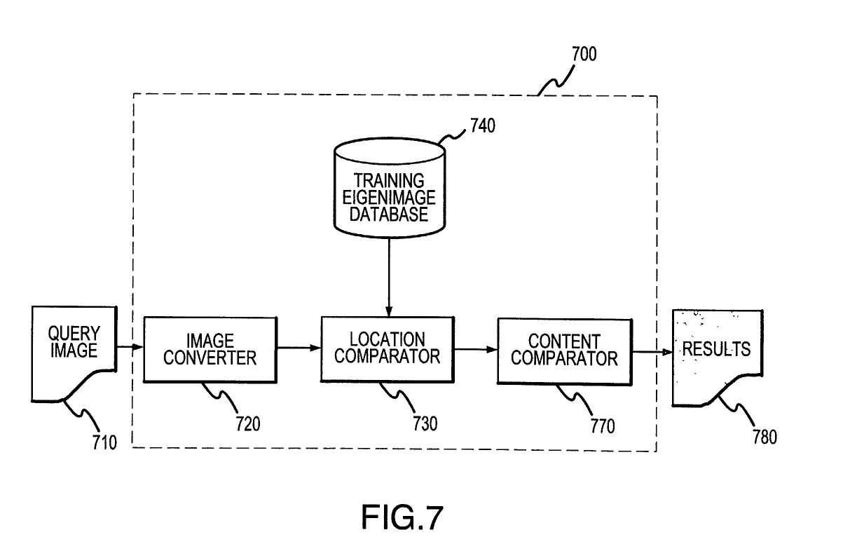

FIG. 7 shows a block diagram of a system embodying one example of the present invention 700. As previously described, before the system can be used, normal and abnormal training images must be selected (not shown) and converted into eigenimages representations (not shown) by an image converter 720. Once the training images have been converted into basis sets of eigeniamges, they are stored in a training eigenimage database 740 that will be accessed by an embodiment of the present invention.

A query image 710 is submitted to the system 700, where it is converted into a query eigenimage by an image converter 720. The query eigenimage is submitted to a location comparator 730 which, using the training eigenimage database 740, identifies the training eigenimages most proximate in location to the area represented in the query eigenimage. The location comparator selects both normal and abnormal training eigenimages from the training eigenimage database 740 as previously described. With the appropriate normal and abnormal training eigenimages identified, a content comparator 770 compares the query eigenimage and the identified training eigenimages. The content comparator 770 generates the results 780 of the analysis, indicating whether the original query image represents a normal or abnormal condition for image summarization or retrieving the closest matching image or images from the training set database for decision support. The results 780 may be in the form of a displayed image, a hardcopy report, or another form. Each of the subsystems shown in FIG. 7 operate in accordance with the corresponding methods previously described.

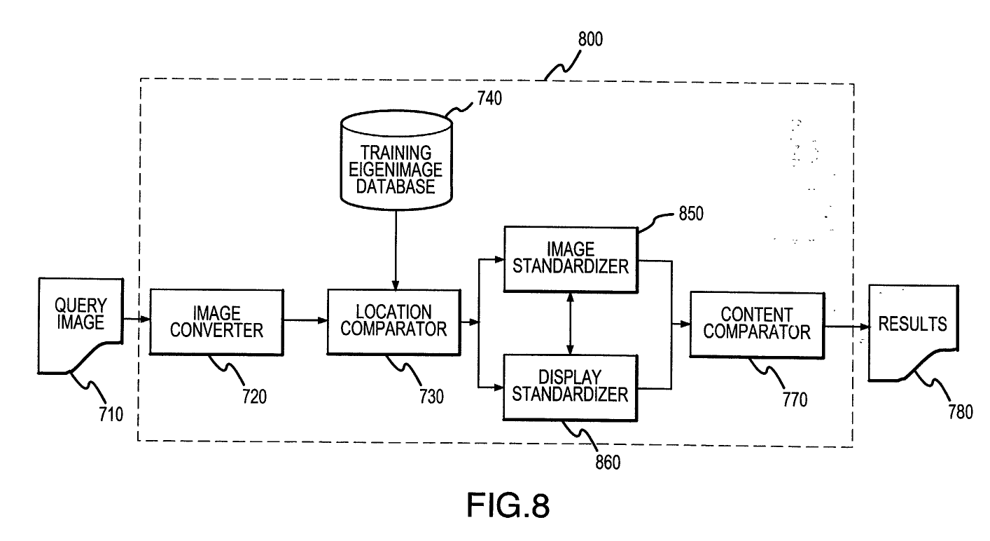

FIG. 8 shows an additional embodiment of a system 800 of the present invention. The system 800 comprises includes the same components used in the ultrasonic system 700 of FIG. 7. Therefore, in the interest of brevity, these components have been provided with the same reference numerals, and an explanation of their functions and operations will not be repeated. The main difference between the system 800 depicted in FIG. 8 and the system 700 depicted in FIG. 7 is that the system 800 incorporates an image standardizer 850 and a display standardizer 860. In a system where, for example, contrast insensitive analyses of the images are used as previously described, the image standardizer 850 might not be necessary. However, if contrast specific comparison analyses are made, the training eigenimages selected from the training eigenimage database 740 by the location comparator 730 will have to be standardized in contrast and/or intensity as previously described. The image standardizer 850 would perform these standardizing functions. Comparably, the display standardizer 860 would standardize the training eigenimages selected from the training eigenimage database 740 by the location comparator 730 for scale and orientation as previously described. Once the training eigenimages have been standardized, they are ready to be compared to the query eigenimage by the content comparator 770, which will generate the results 780 of the analysis.

It is to be understood that, even though various embodiments and advantages of the present invention have been set forth in the foregoing description, the above disclosure is illustrative only. Changes may be made in detail, and yet remain within the broad principles of the invention. For example, although the disclosed embodiments employ particular processes to standardize intensity of the images, different image intensity standardization processes could be used, or uniform image acquisition could be used in gathering the training set images and the query images to eliminate this process. Similarly, a process other than the use of the AIR program could be used to standardize the orientation and scale of the images, or uniform image acquisition could be used to eliminate the need for such standardization.

1. A method for classifying a query image of a query area as normal or abnormal, the method comprising
selecting a plurality of training images of a plurality of normal training images representing normal conditions in a region of interest, and a plurality of abnormal training images representing abnormal conditions in the region of interest;
representing the normal training images as a normal eigenimages;
representing the abnormal training images as an abnormal eigenimages;
representing the query image by a query eigenimage;
choosing normal comparison eigenimages representing an area of interest closest to the query area from the normal eigenimages and abnormal comparison eigenimages representing an area of interest closest to the query area from the abnormal eigenimages; and
classifying the query image as normal when the query eigenimage most closely compares with normal comparison eigenimages and classifying the query image as abnormal when the query eigenimage most closely compares with abnormal comparison eigenimages.

2. The method of claim 1 further comprising standardizing at least one image property of the training images.

3. The method of claim 2 wherein the image property is intensity.

4. The method of claim 2 wherein the image property is contrast.

5. The method of claim 1 further comprising standardizing at least one display property of the training images.

6. The method of claim 5 wherein the display property is scale.

7. The method of claim 5 wherein the display property is orientation.

8. The method of claim 1 further comprising manually classifying a plurality of unclassified training images into normal training images and abnormal training images.

9. The method of claim 1 further comprising not classifying the query image when no normal eigenimages and no abnormal eigenimages represent an area suitably close to the query area.

10. The method of claim 1 further comprising adding the query image to the plurality of training images after the query image has been classified.

11. A method for identifying a comparison image for comparison with a query image of a query area, the method comprising:
selecting a plurality of training images of normal training images representing normal and abnormal conditions in a region of interest;
representing the training images as training eigenimages;
representing the query image by a query eigenimage;
identifying a comparison eigenimage, the comparison eigenimage being a training eigenimage that most closely compares with the query eigenimage; and
identifying from the training images the comparison image, the comparison image being a training image representative of an image area equivalent to the query area and most closely comparing with the query image in substantive image attributes.

12. The method of claim 11 further comprising identifying a plurality of comparison eigenimages that most closely compare with the query eigenimage and identifying from the training images a plurality of comparison images, the comparison images being training images representative of an image area equivalent to the query area and most closely comparing with the query image in substantive image attributes.

13. The method of claim 11 further comprising standardizing at least one image property of the training images.

14. The method of claim 13 wherein the image property is intensity.

15. The method of claim 13 wherein the image property is contrast.

16. The method of claim 11 further comprising standardizing at least one display property of the training images.

17. The method of claim 16 wherein the display property is scale.

18. The method of claim 16 wherein the display property is orientation.

19. The method of claim 11 further comprising manually classifying a query image as normal or abnormal.

20. The method of claim 19 further comprising adding the query image to the plurality of training images after the query image has been classified.

21. A method for classifying a query image of a query area as normal or abnormal compared with a plurality of training images classified as normal training images and abnormal training images, the method comprising:
representing the normal training images as normal eigenimages;
representing the abnormal training images as abnormal eigenimages;
representing the query image into a query eigenimage;
identifying normal comparison eigenimages representing an area of interest closest to the query area and abnormal comparison eigenimages representing an area of interest closest to the query area; and
classifying the query image as normal when the query eigenimage most closely resembles normal eigenimages and classifying the query image as abnormal when the query eigenimage most closely resembles abnormal eigenimages.

22. The method of claim 21 further comprising standardizing at least one image property of the training images.

23. The method of claim 22 wherein the image property is intensity.

24. The method of claim 22 wherein the image property is contrast.

25. The method of claim 21 further comprising standardizing at least one display property of the training images.

26. The method of claim 25 wherein the display property is scale.

27. The method of claim 25 wherein the display property is orientation.

28. The method of claim 21 further comprising manually classifying a plurality of unclassified training images into normal training images and abnormal training images.

29. The method of claim 21 further comprising not classifying the query image when no normal eigenimages and abnormal eigenimages represent an area suitably close to the query area.

30. The method of claim 29 further comprising adding the query image to the training images after the query image has been classified.

31. A method for identifying from a plurality of training classified as normal training images and abnormal training images a comparison image for comparison with a query image of a query area, the method comprising:
representing the training images as training eigenimages;
representing the query image into a query eigenimage;
identifying a comparison eigenimage, the comparison eigenimage being a training eigenimage that most closely compares with the query eigenimage; and
identifying from the training images the comparison image, the comparison image being a training image representative of an image area equivalent to the query area and most closely comparing with the query image in substantive image attributes.

32. The method of claim 34 further comprising identifying a plurality of comparison eigenimages that most closely compare with the query eigenimage and retrieving from the training images a plurality of comparison images, the comparison images being a plurality of training images from which the plurality of comparison eigenimages were derived.

33. The method of claim 31 further comprising standardizing at least one image property of the training images.

34. The method of claim 33 wherein the image property is intensity.

35. The method of claim 33 wherein the image property is contrast.

36. The method of claim 31 further comprising standardizing at least one display property of the training images.

37. The method of claim 36 wherein the display property is scale.

38. The method of claim 36 wherein the display property is orientation.

39. The method of claim 31 further comprising manually classifying a query image as normal or abnormal.

40. The method of claim 39 further comprising adding the query image to the plurality of training images after the query image has been classified.

41. A classifying system for classifying a query image of a query area as normal or abnormal, the classifying system comprising:
a plurality of training images of normal training images representing normal conditions in a region of interest, and a plurality of abnormal training images representing abnormal conditions in the region of interest;
an image converter, the image converter representing each of the normal training images as normal eigenimages, representing each of the abnormal training images as abnormal eigenimages, and representing the query image as a query eigenimage;
a training eigenimage database, storing the normal eigenimages and the abnormal eigenimages generated by the image converter;
a location comparator, receptive of the query eigenimage generated by the image converter and operably connected with the training eigenimage database, the location comparator choosing from the training eigenimage database normal comparison eigenimages and abnormal comparison eigenimages representing an area of interest closest to the query area; and
a content comparator receiving the query eigenimage, and receiving from the location comparator the normal comparison eigenimages and the abnormal comparison eigenimages, the content comparator classifying the query image as normal when the query eigenimage most closely resembles normal comparison eigenimages and classifying the query image as abnormal when the query eigenimage most closely resembles abnormal comparison eigenimages.

42. The system of claim 41 further comprising a training image standardizer.

43. The system of claim 42 wherein the image standardizer comprises an intensity standardizer.

44. The system of claim 42 wherein the image standardizer comprises a contrast standardizer.

45. The system of claim 41 further comprising a display property standardizer.

46. The system of claim 45 wherein the display property standardizer is a scale standardizer.

47. The system of claim 45 wherein the display property standardizer is an orientation standardizer.

48. The system of claim 41 further comprising a training image integrator adding the query image to the plurality of training images after the query image has been classified.

49. An image retrieval system for identifying a comparison image comparable to a query image of a query area, the retrieval system comprising:
a plurality of training images of a region of interest;
an image converter, the image converter representing each of the training images as training eigenimages and the query image as a query eigenimage;
a training eigenimage database, storing the training eigenimages generated by the image converter; and
a comparator coupled with the training eigenimage database and receiving the query eigenimage, the comparator identifying from the training eigenimage database a comparison training eigenimage that most closely compares with the query eigenimage and identifying the comparison image, the comparison image being a training image representative of an image area equivalent to the query area and most closely comparing with the query image in substantive image attributes.

50. The system of claim 49 further comprising a training image standardizer.

51. The system of claim 50 wherein the image standardizer comprises an intensity standardizer.

52. The system of claim 50 wherein the image standardizer comprises a contrast standardizer.

53. The system of claim 49 further comprising a display property standardizer.

54. The system of claim 53 wherein the display property standardizer is a scale standardizer.

55. The system of claim 53 wherein the display property standardizer is an orientation standardizer.

56. The system of claim 49 further comprising a training image integrator adding the query image to the plurality of training images after the query image has been classified.
